(19) United States
(12) Patent Application Publication
(10) Pub. No.: US 2009/0181992 A1
(43) Pub. Date: Jul. 16, 2009

(54) (S)-PHENYL(HETEROCYCLE)METHANOL-BASED COMPOUNDS, COMPOSITIONS COMPRISING THEM AND METHODS OF THEIR USE

(76) Inventors: Joseph Barbosa, Lambertville, NJ (US); Yingzhi Bi, Plainsboro, NJ (US); Cynthia Anne Fink, Lebanon, NJ (US); Michael Alan Green, Easton, PA (US); Jian Cheng Wang, Dallas, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 12/409,640

(22) Filed: Mar. 24, 2009

Related U.S. Application Data
(63) Continuation of application No. 11/935,040, filed on Nov. 5, 2007.
(60) Provisional application No. 60/857,454, filed on Nov. 7, 2006.

(51) Int. Cl. A61K 31/506; C07D 239/47; A61P 25/00

(52) U.S. Cl. 514/273; 544/297

(57) ABSTRACT

Multicyclic compounds and pharmaceutical compositions comprising them are described, along with methods of their use in the treatment of diseases such as age-associated memory impairment, Alzheimer's disease, Attention-Deficit/Hyperactivity Disorder (ADD/ADHD), autism, Down syndrome, Fragile X syndrome, Huntington's disease, Parkinson's disease, and schizophrenia. Compounds include those of formula I:

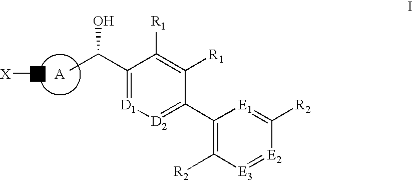

This application is a continuation of U.S. application Ser. No. 11/935,040, filed Nov. 5, 2007, which claims priority to U.S. provisional application No. 60/857,454, filed Nov. 7, 2006, the entireties of which is incorporated herein by reference.

### 1. FIELD OF THE INVENTION

This invention relates to multicyclic compounds, pharmaceutical compositions comprising them, and methods of their use.

### 2. BACKGROUND OF THE INVENTION

The amino acid L-proline reportedly plays a role in regulating synaptic transmission in the mammalian brain. See, e.g., Crump et al., Molecular and Cellular Neuroscience, 13: 25-29 (1999). For example, a synaptosomal biosynthetic pathway of L-proline from ornithine has been reported, and high affinity Na⁺-dependent synaptosomal uptake of L-proline has been observed. Yoneda et al., Brain Res., 239: 479-488 (1982); Balcar et al., Brain Res., 102: 143-151 (1976).

In general, neurotransmitter systems typically have mechanisms that inactivate signaling, many of which work through the action of a Na⁺-dependent transporter. In this case, a Na⁺-dependent transporter for proline has been described, and the molecular entity cloned (SLC6A7 in humans). See, e.g., U.S. Pat. Nos. 5,580,775 and 5,759,788. But the transporter's specific role remains unknown. For example, the human Na⁺-dependent proline transporter is generally localized to synaptic terminals, which is consistent with a role in neurotransmitter signaling. But no high-affinity receptor has been found for proline, suggesting that it is a neuromodulator rather than a neurotransmitter. Shafqat S., et al., Molecular Pharmacology 48:219-229 (1995).

The fact that the Na⁺-dependent proline transporter is expressed in the dorsal root ganglion has led some to suggest that it may be involved in nociception, and that compounds which inhibit the transporter may be used to treat pain. See, e.g., U.S. Patent Application No. 20030152970A1. But this suggestion is not supported by experimental data.

### 3. SUMMARY OF THE INVENTION

This invention encompasses multicyclic compounds, pharmaceutical compositions comprising them, and methods of their use. One embodiment of the invention encompasses a compound of formula I:

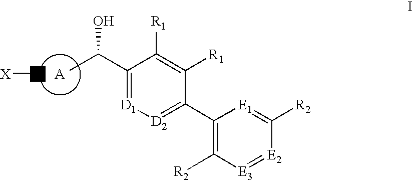

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; X is optionally substituted heteroaryl; each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R_{A} is independently hydrogen or optionally substituted alkyl, aryl, arylalkyl, alkylaryl, heterocycle, heterocycle-alkyl, or alkyl-heterocycle; and each R_{B} is independently hydrogen or optionally substituted alkyl, aryl, arylalkyl, alkylaryl, heterocycle, heterocycle-alkyl, or alkyl-heterocycle.

Preferred compounds inhibit the proline transporter, and particular compounds do so without substantially affecting the dopamine or glycine transporters.

Another embodiment of the invention encompasses pharmaceutical compositions of the various compounds described herein.

Another embodiment encompasses methods of improving cognitive performance and of treating, managing and/or preventing various diseases and disorders using compounds of the invention.

### 4. DETAILED DESCRIPTION OF THE INVENTION

This invention is based, in part, on the discovery that the proline transporter encoded by the human gene at map location 5q31-q32 (SLC6A7 gene; GENBANK accession no. NM_{—}014228) can be a potent modulator of mental performance in mammals. In particular, it has been found that genetically engineered mice that do not express a functional product of the murine ortholog of the SLC6A7 gene display significantly increased cognitive function, attention span, learning, and memory relative to control animals. See U.S. patent application Ser. Nos. 11/433,057 and 11/433,626, both filed May 12, 2006.

In view of this discovery, the protein product associated with the SLC6A7 coding region was used to discover compounds that may improve cognitive performance and may be useful in the treatment, prevention and/or management of diseases and disorders characterized, at least in part, by loss of cognitive, learning and/or memory function.

### 4.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “DTIC₅₀” means an IC₅₀ against human recombinant dopamine transporter as determined using the assay described in the Examples, below.

Unless otherwise indicated, the term “GTIC₅₀” means an IC₅₀ for human recombinant glycine transporter as determined using the assay described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the term “potent proline transporter inhibitor” means a compound that has a PTIC₅₀ of less than about 200 nM.

Unless otherwise indicated, the terms “prevent,” “preventing” and “prevention” contemplate an action that occurs before a patient begins to suffer from the specified disease or disorder, which inhibits or reduces the severity of the disease or disorder, or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound is an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, the term “PTIC₅₀” means an IC₅₀ for human recombinant Na⁺-dependent proline transporter as determined using the assay described in the Examples, below.

Unless otherwise indicated, the term “potent proline transporter inhibitor” means a compound that has a PTIC₅₀ of less than about 200 nM.

Unless otherwise indicated, the term “stereomerically enriched composition of” a compound refers to a mixture of the named compound and its stereoisomer(s) that contains more of the named compound than its stereoisomer(s). For example, a stereoisomerically enriched composition of (S)-butan-2-ol encompasses mixtures of (S)-butan-2-ol and (R)-butan-2-ol in ratios of, e.g., about 60/40, 70/30, 80/20, 90/10, 95/5, and 98/2.

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 4.2. Compounds of the Invention

This invention encompasses compounds of formula I:

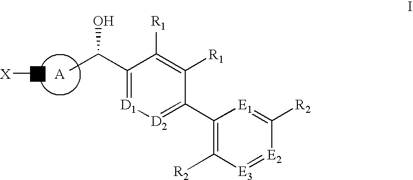

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; X is optionally substituted heteroaryl; each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R_{A} is independently hydrogen or optionally substituted alkyl, aryl, arylalkyl, alkylaryl, heterocycle, heterocycle-alkyl, or alkyl-heterocycle; and each R_{B} is independently hydrogen or optionally substituted alkyl, aryl, arylalkyl, alkylaryl, heterocycle, heterocycle-alkyl, or alkyl-heterocycle.

In one embodiment, A is monocyclic. In another, A is bicyclic. In another, A is unsubstituted. In another, A is optionally substituted pyrrolidine, piperidine, hexahydropyrimidine, 1,2,3,6-tetrahydropyridine, octahydrocyclopenta[c]pyrrole, or octahydropyrrolo[3,4-c]pyrrole.

In one embodiment, one of D₁ and D₂ is N. In another, both D₁ and D₂ are N. In another, both D₁ and D₂ are CR₁.

In one embodiment, one of E₁, E₂ and E₃ is N. In another, two of E₁, E₂ and E₃ are

N. In another, all of E₁, E₂ and E₃ are N. In another, all of E₁, E₂ and E₃ are independently CR₂.

In one embodiment, R₁ is hydrogen, halogen, or optionally substituted alkyl. In another, R₁ is OR_{A} and R_{A} is, for example, hydrogen or optionally substituted alkyl.

In one embodiment, R₂ is hydrogen, halogen, or optionally substituted alkyl. In another, R₂ is OR_{A} and R_{A} is, for example, hydrogen or optionally substituted alkyl.

In one embodiment, X is an optionally substituted 5-, 6-, 9- or 10-membered heteroaryl. In another, X is optionally substituted 5- or 6-membered heteroaryl. In another, X is of the formula:

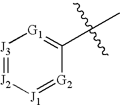

wherein: each of G₁ and G₂ are independently N or CR₃; each of J₁, J₂ and J₃ are independently N or CR₄; each R₃ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; and each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; provided that at least one of J₁, J₂ and J₃ is CR₄.

In a particular embodiment, one of G₁ and G₂ is N. In another, both G₁ and G₂ are N. In another, both G₁ and G₂ are CR₃. In another, one of J₁, J₂ and J₃ is N. In another, two of J₁, J₂ and J₃ are N. In another, all of J₁, J₂ and J₃ are independently CR₄.

In one embodiment, R₃ is hydrogen, halogen, or optionally substituted alkyl. In another, R₃ is OR_{A} and R_{A} is, for example, hydrogen or optionally substituted alkyl.

In one embodiment, R₄ is hydrogen, halogen, or optionally substituted alkyl. In another, R₄ is OR_{A} and R_{A} is, for example, hydrogen or optionally substituted alkyl.

One embodiment of the invention encompasses compounds of formula I(A):

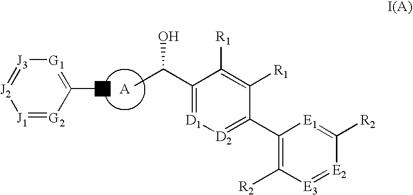

and pharmaceutically acceptable salts and solvates thereof.

Another encompasses compounds of formula I(B):

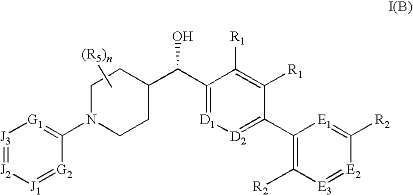

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and n is 0-5.

Another encompasses compounds of formula I(C):

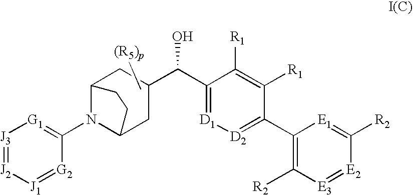

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and p is 0-7.

Another encompasses compounds of formula I(D):

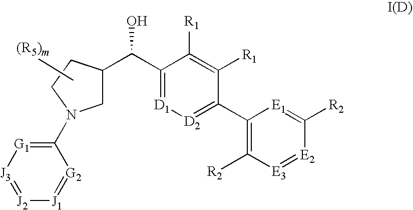

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and m is 0-4.

This invention encompasses stereomerically pure compounds and stereomerically enriched compositions of them. Stereoisomers may be asymmetrically synthesized or resolved using standard techniques such as chiral columns, chiral resolving agents, or enzymatic resolution. See, e.g., Jacques, J., et al., Enantiomers, Racemates and Resolutions (Wiley Interscience, New York, 1981); Wilen, S. H., et al, Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions, p. 268 (E. L. Eliel, Ed., Univ. of Notre Dame Press, Notre Dame, Ind., 1972).

Examples of compounds encompassed by the invention include:
(S)-2-(4-((3′-chlorobiphenyl-4-yl)(hydroxy)methyl)piperidin-1-yl)pyrimidin-5-ol;
(S)-(3′-chlorobiphenyl-4-yl)(1-(pyrimidin-2-yl)piperidin-4-yl)methanol;
(S)-(1-(pyrimidin-2-yl)piperidin-4-yl)(4′-(trifluoromethyl)biphenyl-4-yl)methanol;
(S)-(5′-chloro-2′-fluorobiphenyl-4-yl)(8-(pyrimidin-2-yl)-8-azabicyclo[3.2.1]octan-3-yl)methanol;
(S)-biphenyl-4-yl-(1-pyrimidin-2-yl-1,2,3,6-tetrahydro-pyridin-4-yl)-methanol;
(S)-(1-(pyrimidin-2-yl)piperidin-4-yl)(2′,3,4′-trifluorobiphenyl-4-yl)methanol;
(S)-(3′-chloro-3-methylamino-biphenyl-4-yl)-(1-pyrimidin-2-yl-piperidin-4-yl)-methanol;
(S)-(3-amino-3′-chlorobiphenyl-4-yl)(1-(pyrimidin-2-yl)piperidin-4-yl)methanol;
(S)—N-(3′-chloro-4-(hydroxy(1-(pyrimidin-2-yl)piperidin-4-yl)methyl)biphenyl-3-yl)acetamide;
(S)—N-{3′-chloro-4-[hydroxyl-(1-pyrimidin-2-yl-piperidin-4-yl)-methyl]-biphenyl-3-yl}-acetamide;
(S)-3′-chloro-4-[hydroxy-(1-pyrimidin-2-yl-piperidin-4-yl)-methyl]-biphenyl-3-ol; and
(S)-(3′-chloro-3-methoxy-biphenyl-4-yl)-(1-pyrimidin-2-yl-piperidin-4-yl)-methanol.

Preferred compounds of the invention are potent proline transporter inhibitors. Particular potent proline transporter inhibitors have a PTIC₅₀ of less than about 150, 125, 100, 75, 50 or 25 nM.

Some compounds inhibit the murine Na⁺-dependent proline transporter, as determined by the method described in the Examples below, with an IC₅₀ of less than about 150, 125, 100, 75, 50 or 25 nM.

Some compounds do not significantly inhibit the dopamine transporter. For example, some potent proline transporter inhibitors inhibit the dopamine transporter with an IC₅₀ of greater than about 0.5, 1, 2.5, 5, or 10 μM as determined using the assay described in the Examples below.

Some compounds do not significantly inhibit the glycine transporter. For example, some potent proline transporter inhibitors inhibit the glycine transporter with an IC₅₀ of greater than about 0.5, 1, 2.5, 5, or 10 μM as determined using the assay described in the Examples below.

### 4.3. Preparation of Compounds

Compounds of the invention may be obtained or prepared using synthetic methods known in the art (see, e.g., U.S. patent application Ser. Nos. 11/433,057 and 11/433,626, both filed May 12, 2006), as well as those described herein. For example, various piperidine-based compounds can be prepared by reducing the product formed by the general approach shown below in Scheme I:

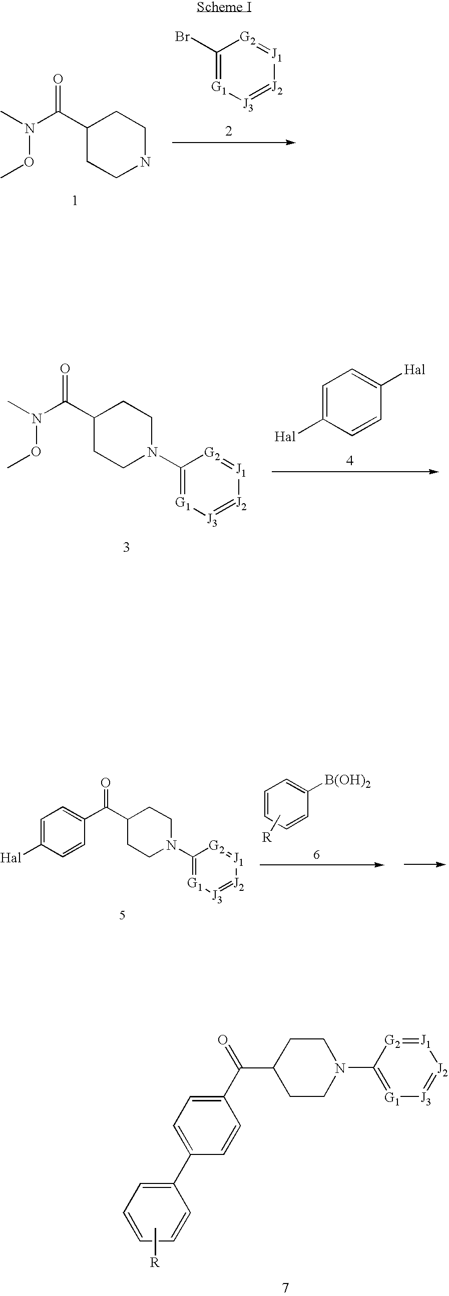

In this approach, a compound of formula 1 (e.g., as a TFA salt) is contacted with a compound of formula 2 (G₁, G₂, J₁, J₂ and J₃ are defined herein) under suitable conditions to provide compound 3. Suitable conditions include, for example, TEA and heat. Compound 3 is then contacted with compound 4 under suitable conditions to provide compound 5. Here, suitable conditions include, for example, n-BuLi in THF. Compound 5 is then contacted with a compound of formula 6 to provide compound 7. Here, suitable conditions include, for example, Pd(Ph₃P)₄, K₃PO₄, DME, water and heat.

Compounds of formula 7 can be reduced under suitable conditions (e.g., sodium borohydride) to provide compounds of formula 8, as shown below in Scheme II:

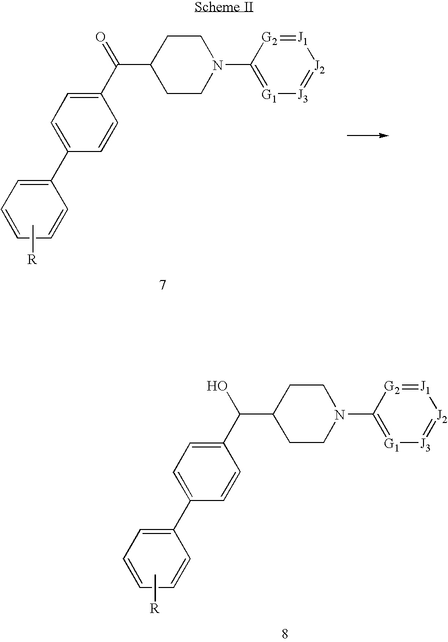

Stereoisomers of compounds of formula 8 can be resolved by conventional means (e.g., chromatography or formation of chiral salts).

Some specific reaction conditions that can be used in the various synthetic schemes shown above are provided in the Examples, below.

### 4.4. Methods of Treatment

One embodiment of this invention encompasses a method of inhibiting a proline transporter, which comprises contacting a proline transporter (in vitro or in vivo) with a sufficient amount of a compound of the invention. Preferred proline transporters are encoded by the human gene SLC6A7, the murine ortholog thereof, or a nucleic acid molecule that encodes a proline transporter and that hybridizes under standard conditions to the full length of either.

Another embodiment encompasses a method of improving the cognitive performance of a human patient, which comprises administering to the patient an effective amount of a compound of the invention. Examples of improved cognitive performance include enhanced learning (e.g., learning more quickly), improved comprehension, improved reasoning, and improved short- and/or long-term memory.

Another embodiment encompasses a method of treating, managing or preventing a cognitive disorder (e.g., difficulty in thinking, reasoning, or problem solving), memory loss (short- and long-term), or a learning disorder (e.g., dyslexia, dyscalculia, dysgraphia, dysphasia, dysnomia), which comprises administering to the patient an effective amount of a compound of the invention.

Another embodiment encompasses a method of treating, managing or preventing a disease or disorder, or a cognitive impairment associated therewith, in a human patient, which comprises administering to the patient a therapeutically or prophylactically effective amount of a compound of the invention. Examples of diseases and disorders include age-associated memory impairment, Alzheimer's disease, Attention-Deficit/Hyperactivity Disorder (ADD/ADHD), autism, Down syndrome, Fragile X syndrome, Huntington's disease, Parkinson's disease, and schizophrenia. Additional disorders include adverse sequelae of brain damage caused by, for example, oxygen starvation, traumatic injury, heart attack or stroke.

The invention also encompasses methods of treating, preventing and managing dementia, including dementia associated with metabolic-toxic, structural and/or infectious causes.

Metabolic-toxic causes of dementia include: anoxia; B₁₂ deficiency; chronic drug, alcohol or nutritional abuse; folic acid deficiency; hypercalcemia associated with hyperparathyroidism; hypoglycemia; hypothyroidism; organ system failure (e.g., hepatic, respiratory, or uremic encephalopathy); and pellagra.

Structural causes of dementia include: amyotrophic lateral sclerosis; brain trauma (e.g., chronic subdural hematoma, dementia pugilistica); brain tumors; cerebellar degeneration; communicating hydrocephalus; irradiation to frontal lobes; multiple sclerosis; normal-pressure hydrocephalus; Pick's disease; progressive multifocal leukoencephalopathy; progressive supranuclear palsy; surgery; vascular disease (e.g., multi-infarct dementia); and Wilson's disease.

Infectious causes of dementia include: bacterial endocarditis; Creutzfeldt-Jakob disease; Gerstmann-Straussler-Scheinker disease; HIV-related disorders; neurosyphilis; tuberculous and fungal meningitis; and viral encephalitis.

### 4.5. Pharmaceutical Compositions

This invention encompasses pharmaceutical compositions and dosage forms comprising compounds of the invention as their active ingredients. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

### 5. EXAMPLES

### 5.1. Preparation of (S)-(3′-Chlorobiphenyl-4-yl)(1-(pyrimidin-2-yl)piperidin-4-yl)methanol

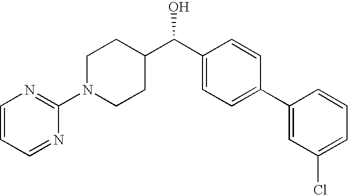

The title compound was isolated from (S/R)-(3′-chlorobiphenyl-4-yl)(1-(pyrimidin-2-yl)piperidin-4-yl)methanol. The racemic mixture was prepared from (3′-chlorobiphenyl-4-yl)(1-(pyrimidin-2-yl)piperidin-4-yl)methanone.

A. (3′-Chlorobiphenyl-4-yl)(1-(pyrimidin-2-yl)piperidin-4-yl)methanone: 3-Chlorophenyl boronic acid (Alfa Aesar, purity 97%) (40.7 g, 261.19 mmol, 1.4 eq) was dissolved in isopropanol (Aldrich, ACS reagent grade) (800 ml) under nitrogen atmosphere. This was added to a solution of aqueous potassium carbonate (77 g in 150 ml water), bis(triphenylphosphine)palladium(II) dichloride (PdCl₂(PPh₃)₂) (0.65 g, 0.93 mmol, 0.5 mol. eq.) and (4-bromophenyl)(piperidine-4-yl)methanone (50 g, 187 mmol, 1 eq) were stirred at 80° C. for three hours and deemed complete by LC/MS. After the reaction mixture cooled down to 50° C., it was filtered through celite pad, washed with methanol (1 liter). The filtrate was diluted with water (200 ml), then the organic solvent removed under reduced pressure. The resulting crude product was dissolved in ethyl acetate (800 ml) and washed with 1N sodium hydroxide (2×40 ml) and water (1×40 ml).

The organic layer was stirred with aqueous lactic acid (64 g of 85% lactic acid in 600 ml of water) at 50° C. for 20 minutes. After the organic layer was separated (solution assay indicated 8% of product present in the organic layer, which can be captured by additional lactic acid extraction), the aqueous layer was washed with ethyl acetate (2×100 ml). The aqueous layer was separated, basified to pH=11 with 25% NaOH (˜70 ml), and then extracted with ethyl acetate (2×200 ml), dried over sodium sulfate, filtered and concentrated under reduced pressure to obtained biaryl product 46.23 g (83%) as a syrup. HPLC indicated 99.4% product and 0.57% of debrominated staring material.

The above product was dissolved in mixture of ethyl acetate (900 ml) and ethanol (45 ml) and heated at 50° C. 6M aq. HCl (40 ml) was added dropwise over a period of ten minutes. After 20 minutes, the reaction mixture was cooled to room temperature, and stirring was continued for an additional hour. The resulting white solid was filtered and dried under vacuum at 50° C. for five hours to afford 49.8 grams of the biaryl HCl salt (80%). HPLC indicated pure product. ¹H NMR (DMSO-d₆) δ: 1.92 (m, 4H), 2.52 (m, 2H), 3.12 (m, 2H), 3.82 (m, 1H), 7.51 (m, 2H), 7.75 (m, 1H), 7.82 (br s, 1H), 7.92 (bs d, 2H), 8.12 (brd, 2H), 9.0 (br s, 2H). MH⁺=300, 302 (about 3:1).

B. (S/R)-(3′-Chlorobiphenyl-4-yl)(1-(pyrimidin-2-yl)piperidin-4-yl)methanol: To a solution of biphenyl-4-yl-(1-pyrimidin-2-yl-1,2,3,6-tetrahydro-pyridin-4-yl)-methanone (12.2 mg, 0.0355 mmol) in methanol (0.5 ml), was added CeCl₃ heptahydrate (13.2 mg, 0.0355 mmol) and sodium borohydride (1.5 mg, 0.0355 mmol) at room temperature. The mixture was stirred for 1 hour and diluted with EtOAc (10 ml). The mixture was washed with water (5 ml), brine (5 ml), dried (MgSO₄), filtered, and concentrated under reduced pressure to furnish the crude product. This material was purified by column chromatography (6% MeOH/CH₂Cl₂) to give (S/R)-(3′-chlorobiphenyl-4-yl)(1-(pyrimidin-2-yl)piperidin-4-yl)methanol (12 mg, 98%) as a white gel: ¹H NMR (CDCl₃, 400 MHz) δ 8.36 (d, J=6.4 Hz, 2H), 7.62-7.37 (m, 9H), 6.46 (t, J=6.4 Hz, 1H), 6.02 (m, 1H), 5.24 (m, 1H), 4.31 (m, 2H), 3.96 (m, 1H), 3.83 (m, 1H), 2.14 (m, 2H); MS calc'd for C₂₂H₂₂N₃O [M+H]⁺: 344; Found: 344.

C. (S)-(3′-Chlorobiphenyl-4-yl)(1-(pyrimidin-2-yl)piperidin-4-yl)methanol: About 1.1 grams of the racemic product was dissolved in 80 ml of 60% ethanol in hexanes. The enantiomers were separated by normal phase chiral chromatography at ambient temperature using ChiralPak AD-H, 20×250 mm column: flow=7 ml/min.; inj. vol. 8 ml, detection at 220 nm. The title compound eluted at 25 minutes. Ten injections were made to prepare the entire sample.

### 5.2. Preparation of (S)-2-(4-((3′-Chlorobiphenyl-4-yl)(hydroxy)methyl)piperidin-1-yl)pyrimidin-5-ol

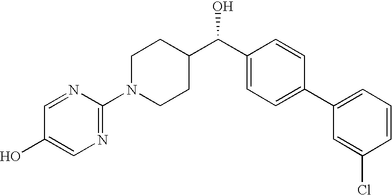

The title compound is isolated by separating the enantiomers of (S/R)-2-(4-((3′-chlorobiphenyl-4-yl)(hydroxy)methyl)piperidin-1-yl)pyrimidin-5-ol. The racemic mixture was prepared from (3′-chlorobiphenyl-4-yl)(1-(4-hydroxylpyrimidin-2-yl)piperidin-4-yl)methanone.

A. (3′-Chlorobiphenyl-4-yl)(1-(4-methoxypyrimidin-2-yl)piperidin-4-yl)methanone: A suspension of (3′-chlorobiphenyl-4-yl)(piperidin-4-yl)methanone (0.44 g, 1.31 mmol), 1-chloro-4-methoxypyrimidine (0.19 g, 1.31 mmol), triethylamine (0.36 ml, 1.32 mmol) and acetonitrile (3 ml) was microwaved at 200° C. for 52 minutes. The mixture was cooled and concentrated in vacuo. To the residue was added methylene chloride (50 ml) and the organic phase was washed with brine, a saturated solution of sodium bicarbonate, dried over magnesium sulfate, and concentrated. The residue was purified by flash chromatography (SiO₂: methylene chloride) to yield 0.20 g of (3′-chlorobiphenyl-4-yl)(1-(4-methoxypyrimidin-2-yl)piperidin-4-yl)methanone as a clear oil. The spectral data was consistent with structure: ¹H NMR (CDCl₃): δ 8.05 (2H, s), 7.95 (2H, m). 7.43 (6H, m), 4.65 (2H, d), 3.74 (3H, s), 3.48 (1H, m), 3.03 (2H, m), 1.77 (H, m). MS (M+1)=408.

B. (3′-Chlorobiphenyl-4-yl)(1-(4-hydroxylpyrimidin-2-yl)piperidin-4-yl)methanone: To a solution of (3′-chlorobiphenyl-4-yl)(1-(4-methoxypyrimidin-2-yl)piperidin-4-yl)methanone (0.20 g, 0.52 mmol) in methylene chloride (30 ml) cooled to 0° C. was added a 1.0M solution of boron tribromide in methylene chloride (2.06 ml, 2.06 mmol). The mixture was stirred for 30 minutes and then an additional 30 minutes at room temperature and then poured over ice. The pH of the solution was adjusted to 6 and the layers were separated. The organic phase was washed with brine, dried over magnesium sulfate and concentrated to yield a brown oil. The oil was purified by flash chromatography (SiO₂: 2% methanol/methylene chloride to give (3′-chlorobiphenyl-4-yl)(1-(4-hydroxylpyrimidin-2-yl)piperidin-4-yl)methanone as a clear foam 0.10 g. Spectral data was consistent with structure. ¹H NMR (CDCl₃): δ 8.23 (2H, s), 7.97 (2H, d). 7.36 (6H, m), 4.60 (2H, d), 3.48 (1H, t), 3.03 (2H, m), 1.83 (4H, m). MS (M+1)=394.

C. (S/R_{B}-2-(4-((3′-chlorobiphenyl-4-yl)(hydroxy)methyl)piperidin-1-yl)pyrimidin-5-ol: To a solution of (3′-chlorobiphenyl-4-yl)(1-(4-hydroxylpyrimidin-2-yl)piperidin-4-yl)methanone (0.10 g, 0.25 mmol) in methanol (5 ml) was added sodium borohydride (0.10 g, 2.7 mmol) portionwise. The mixture was stirred for 30 minutes and then concentrated in vacuo. To the concentrate was added water (5 ml), and then mixture was acidified to pH 6 with 1 N hydrochloric acid. The solid precipitate was collected, washed with water and dried under vacuum to yield 42 mg of (S/R)-2-(4-((3′-chlorobiphenyl-4-yl)(hydroxy)methyl)piperidin-1-yl)pyrimidin-5-ol as a white solid. Spectral data was consistent with structure. ¹H NMR (DMSO): δ 9.10 (1H, s), 7.99 (2H, s). 7.65 (4H, m), 7.43 (4H, m), 5.23 (1H, d), 4.51 (2H, dd), 4.34 (1H, t), 2.67 (2H, q), 1.75 (2H, m). 1.32 (3H, m). MS (M+1)=396.

D. (S)-2-(4-((3′-chlorobiphenyl-4-yl)(hydroxy)methyl)piperidin-1-yl)pyrimidin-5-ol: The racemic compound is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.3. Preparation of (S)-(1-(Pyrimidin-2-yl)piperidin-4-yl)(4′-(trifluoromethyl)biphenyl-4-yl)methanol

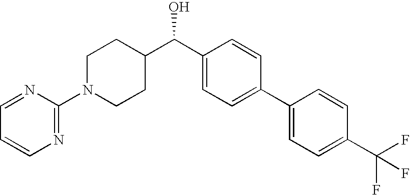

The title compound is isolated by separating the enantiomers of (S/R)-(1-(pyrimidin-2-yl)piperidin-4-yl)(4′-(trifluoromethyl)biphenyl-4-yl)methanol. The racemic mixture was prepared from (1-(pyrimidin-2-yl)piperidin-4-yl)(4-4-trifluoromethylphenyl)-phenyl)methanone, which was prepared from (4-bromophenyl)(1-(pyrimidin-2-yl)piperidin-4-yl)methanone as described in steps A-D below.

A. N-Methoxy-N-methylpiperidine-4-carboxamide: A mixture of N-tert-butoxycarbonyl isonipecotic acid (1.50 g, 6.54 mmol, 1 eq), 1-(3-dimethylaminopropyl)-3-ethylcarbodiimide hydrochloride (1.88 g, 9.81 mmol, 1.5 eq), 1-hydroxybenzotriazole (1.33 g, 9.81 mmol, 1.5 eq), and N,N-dimethylformamide (26 ml) was treated with N,N-diisopropylethylamine (4.60 ml, 26.2 mmol, 4 eq). The resultant yellow solution was stirred at room temperature for 5 minutes, and then N,O-dimethylhydroxylamine hydrochloride (766 mg, 7.85 mmol, 1.2 eq) was added, and stirring continued for 92 hours. The reaction mixture was diluted with 100 ml of ethyl acetate and washed sequentially with 1 N aq. NaOH, 1 N aq. HCl and brine. The organic phase was dried over Na₂SO₄ and concentrated to give an oil which was used with no further purification.

This oil was dissolved in 1:2 trifluoroacetic acid/dichloromethane (9 ml), and the reaction mixture was stirred at ambient temperature for 17 hours and then concentrated. Ether (30 ml) was added and the white solid which formed was collected by filtration, washed with ether and dried to afford 1.50 g (80% yield, 2 steps) of analytically pure product: 400 MHz ¹H NMR (d₆-DMSO): 8.55 (br s, 1H), 8.25 (br s, 1H), 3.69 (s, 3H), 3.31 (m, 2H), 3.10 (s, 3H), 2.98 (m, 3H), 1.65-1.84 (m, 4H).

B. N-Methoxy-N-methyl-1-(pyrimidin-2-yl)piperadine-4-carboxamide: A mixture of N-methoxy-N-methylpiperidine-4-carboxamide (1.50 g, 5.25 mmol, 1 eq), 2-chloropyrimidine (634 mg, 5.25 mmol, 1 eq), triethylamine (2.20 ml, 15.8 mmol, 3 eq), and ethanol (21 ml) was heated at 100° C. in a sealed tube for 19 hours. The reaction mixture was allowed to cool to room temperature and then concentrated. The residue was dissolved in dichloromethane, washed with water and brine, dried over Na₂SO₄, and concentrated. Column chromatography (silica gel, 50%→60% ethyl acetate/hexanes) gave 1.28 g (97% yield) of the product as a colorless oil: HPLC: 100% pure at 1.905 min (YMC-Pack ODS-A 4.6×33 mm column, 0%→100% solvent B over 4 min, 3 ml/min, 220 nm); LCMS (M+H)⁺=251.05; 400 MHz ¹H NMR (CDCl₃) 8.29 (d, J=4.7 Hz, 2H), 6.45 (t, J=4.7 Hz, 1H), 4.80 (m, 2H), 3.73 (s, 3H), 3.19 (s, 3H), 2.95 (m, 3H), 1.70-1.84 (m, 4H).

C. (4-Bromophenyl)(1-(pyrimidin-2-yl)piperidin-4-yl)methanone: A solution of 1,4-dibromobenzene (2.29 g, 9.72 mmol, 1.9 eq) in THF (20 ml) under N₂ was cooled to −78° C., and n-butyllithium (1.6 M in hexanes, 4.8 ml, 7.67 mmol, 1.5 eq) was added dropwise. The reaction mixture was stirred at −78° C. for 40 minutes, and a solution of N-methoxy-N-methyl-1-(pyrimidin-2-yl)piperadine-4-carboxamide (1.28 g, 5.11 mmol, 1 eq) in THF (5 ml) was added dropwise via a cannula. After 3 hours at −78° C., the reaction mixture was warmed to 0° C., stirred for 1 hour, and then quenched with 1 N aq. HCl (10 ml). The mixture was diluted with 150 ml of ethyl acetate, washed sequentially with saturated aq. NaHCO₃ and brine (75 ml each), and the organic phase was dried over Na₂SO₄ and concentrated. Column chromatography (silica gel, CH₂Cl₂→3.5% ethyl acetate/CH₂Cl₂) afforded 1.47 g (83% yield) of the product as a pale yellow solid: HPLC: 99% pure at 3.748 min (YMC-Pack ODS-A 4.6×33 mm column, 0%→100% solvent B over 4 min, 3 ml/min, 220 nm); LCMS (M+H)⁺=345.90; 400 MHz ¹H NMR (CDCl₃) 8.31 (d, J=4.7 Hz, 2H), 7.83 (d, J=8.5 Hz, 2H), 7.63 (d, J=8.5 Hz, 2H), 6.48 (t, J=4.7 Hz, 1H), 4.81 (m, 2H), 3.49 (m, 1H), 3.08 (m, 2H), 1.72-1.95 (m, 4H).

D. (1-(Pyrimidin-2-yl)piperidin-4-yl)(4-4-trifluoromethylphenyl)-phenyl)methanone: A mixture of (4-bromophenyl)(1-(pyrimidin-2-yl)piperidin-4-yl)methanone (66 mg, 0.19 mmol, 1 eq), 4-trifluoromethylphenylboronic acid (91 mg, 0.47 mmol, 2.5 eq), potassium phosphate (122 mg, 0.57 mmol, 3 eq), and Pd(PPh₃)₄ (22 mg, 0.019 mmol, 0.1 eq) in 3:1 DME/water (2 ml) was heated at 80° C. under N₂ for 16 hours. The reaction mixture was cooled to room temperature, poured into 1 N NaOH, and extracted twice with dichloromethane. The combined organic layers were dried over Na₂SO₄ and concentrated. Column chromatography (silica gel, 25% ethyl acetate/hexanes) afforded 58 mg (73% yield) of (1-(pyrimidin-2-yl)piperidin-4-yl)(4-4-trifluoromethylphenyl)-phenyl)methanone as a white solid: HPLC: 97% pure at 4.523 min (YMC-Pack ODS-A 4.6×33 mm column, 0%→100% solvent B over 4 min, 3 ml/min, 220 nm); LCMS (M+H)⁺=412.20; 300 MHz ¹H NMR (CDCl₃) 8.32 (d, J=4.7 Hz, 2H), 8.08 (d, J=8.4 Hz, 2H), 7.70-7.74 (m, 6H), 6.48 (t, J=4.7 Hz, 1H), 4.83 (m, 2H), 3.58 (m, 1H), 3.12 (m, 2H), 1.75-2.01 (m, 4H).

E. (S/R)-(1-(pyrimidin-2-yl)piperidin-4-yl)(4′-(trifluoromethyl)biphenyl-4-yl)methanol: Sodium borohydride (3.0 mg, 0.080 mmol, 1.5 eq) was added to a solution of (1-(pyrimidin-2-yl)piperidin-4-yl)(4-4-trifluoromethylphenyl)phenyl)methanone (22 mg, 0.053 mmol, 1 eq) in 1:1 methanol/dichloromethane. The reaction mixture was stirred at room temperature for 1 hour and then slowly quenched with saturated aq. NaHCO₃. The biphasic mixture was extracted twice with dichloromethane, and the combined organic layers were dried over Na₂SO₄ and concentrated. Preparative TLC (500 μm silica gel, 33% ethyl acetate/hexanes) gave 17 mg (77% yield) of (S/R)-(1-(pyrimidin-2-yl)piperidin-4-yl)(4′-(trifluoromethyl)biphenyl-4-yl)methanol as a white solid: HPLC: 100% pure at 4.285 min (YMC-Pack ODS-A 4.6×33 mm column, 0%→100% solvent B over 4 min, 3 ml/min, 220 nm); LCMS (M+H)⁺=414.10; 300 MHz ¹H NMR (CDCl₃) 8.27 (d, J=4.7 Hz, 2H), 7.69 (s, 4H), 7.59 (d, J=8.3 Hz, 2H), 7.42 (d, J=8.2 Hz, 2H), 6.43 (t, J=4.7 Hz, 1H), 4.71-4.87 (m, 2H), 4.48 (m, 1H), 2.72-2.89 (m, 2H), 1.88-2.11 (m, 3H), 1.19-1.49 (m, 3H).

F. (S)-(1-(pyrimidin-2-yl)piperidin-4-yl)(4′-(trifluoromethyl)biphenyl-4-yl)methanol: (S/R)-(1-(pyrimidin-2-yl)piperidin-4-yl)(4′-(trifluoromethyl)biphenyl-4-yl)methanol is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.4. Preparation of (S)-Biphenyl-4-yl-(1-pyrimidin-2-yl-1,2,3,6-tetrahydro-pyridin-4-yl)-methanol

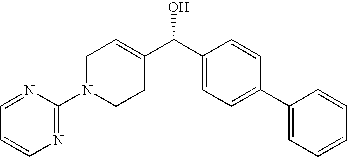

The title compound is isolated by separating the enantiomers of (S/R)-biphenyl-4-yl-(1-pyrimidin-2-yl-1,2,3,6-tetrahydro-pyridin-4-yl)-methanol. The racemic mixture is prepared from biphenyl-4-yl-(1-pyrimidin-2-yl-1,2,3,6-tetrahydro-pyridin-4-yl)-methanone, which was prepared as described in steps A-E below.

A. 1-Pyrimidin-2-yl-piperidin-4-one: To a solution of 2-chloropyrimidine (300 mg, 2.619 mmol) in dioxane (5 ml), was added piperidin-4-one hydrochloride monohydrate (402.3 mg, 2.619 mmol) at room temperature. The mixture was heated at 80° C. overnight and concentrated under reduced pressure. The residue was treated with ethyl acetate (30 ml) and saturated NaHCO₃ (10 ml). After separation of the layers, the aqueous phase was extracted with EtOAc (2×10 ml). The combined organic layers were washed with brine (10 ml), dried (MgSO₄), filtered, and concentrated under reduced pressure to furnish a crude product. This material was purified by column chromatography (40% ethyl acetate/hexanes) to give 1-pyrimidin-2-yl-piperidin-4-one (320 mg, 53%) as an off-white solid: ¹H NMR (CDCl₃, 400 MHz) δ 8.38 (d, J=6.4 Hz, 2H), 6.61 (t, J=6.4 Hz, 9H), 4.16 (t, J=5.6 Hz, 2H), 2.53 (t, J=5.6 Hz, 2H).

B. Triflate: To a solution of LDA (prepared from diisopropylamine (167.4 mg, 1.658 mmol) and n-BuLi (2.5 M in hexanes, 0.663 ml, 1.658 mmol) at −78° C., was added a solution of the above 1-pyrimidin-2-yl-piperidin-4-one (320 mg, 1.382 mmol). The mixture was stirred at the same temperature for 1 hour, followed by the addition of PhNTf₂ (543.1 mg, 1.52 mmol). The reaction mixture was warmed up to room temperature and stirred for 3 hours before it was quenched with the addition of saturated ammonium chloride (15 ml) and ethyl acetate (40 ml). After separation of the layers, the aqueous phase was extracted with ethyl acetate (2×10 ml). The combined organic layers were washed with brine (10 ml), dried (MgSO₄), filtered, and concentrated under reduced pressure to furnish the crude product. This material was purified by column chromatography (20% ethyl acetate/hexanes) to give the corresponding triflate (210.7 mg, 49%) as a white solid as long with recovered starting material (142.9 mg): ¹H NMR (CDCl₃, 400 MHz) δ 8.37 (d, J=6.4 Hz, 2H), 6.59 (t, J=6.4 Hz, 1H), 5.91 (m, 1H), 4.41 (m, 2H), 4.11 (t, J=5.6 Hz, 2H), 2.55 (m, 2H); MS calc'd for C₁₀H11F₃N₃O₃S [M+H]⁺: 310; Found: 310.

C. 1-Pyrimidin-2-yl-1,2,3,6-tetrahydro-pyridin-4-carboxylic Acid Methyl Ester To a solution of the above triflate (210.7 mg, 0.682 mmol) in methanol (10 ml), was added Pd(OAc)₂ (10.7 mg, 0.047 mmol), PPh₃ (31.3 mg, 0.119 mmol) and diisopropyl ethylamine (352.6 mg, 2.728 mmol) at room temperature. Carbon monoxide was bubbled through the solution for 4 hours before the mixture was concentrated under reduced pressure. The residue was treated with ethyl acetate (30 ml) and water (10 ml). The aqueous phase was further extracted with ethyl acetate (2×10 ml). The combined organic layers were washed with brine (10 ml), dried (MgSO₄), filtered, and concentrated under reduced pressure to furnish the crude product. This material was purified by column chromatography (30% ethyl acetate/hexanes) to give 1-pyrimidin-2-yl-1,2,3,6-tetrahydro-pyridin-4-carboxylic acid methyl ester (73.8 mg, 50%) as white crystals: ¹H NMR (CDCl₃, 400 MHz) δ 8.37 (d, J=6.4 Hz, 2H), 7.04 (m, 1H), 6.54 (t, J=6.4 Hz, 1H), 4.41 (m, 2H), 3.98 (t, J=5.6 Hz, 2H), 3.79 (s, 3H), 2.52 (m, 2H).

D. 1-Pyrimidin-2-yl-1,2,3,6-tetrahydro-pyridin-4-carboxylic acid methoxy-methyl amide: To a suspension of 1-pyrimidin-2-yl-1,2,3,6-tetrahydro-pyridin-4-carboxylic acid methyl ester (73.8 mg, 0.337 mmol) and N-methyl-O-methyl hydroxylamine hydrochloride (51.0 mg, 0.552 mmol) in THF (3 ml), was added isopropyl magnesiumchloride (2.0 M in THF, 0.505 ml) at −20° C. over 15 minute-period. The mixture was stirred at −10° C. for another 30 minutes before it was quenched with the addition of saturated ammonium chloride (10 ml). The mixture was extracted with EtOAc (2×15 ml). The combined organic layers were washed with brine (15 ml), dried (MgSO₄), filtered, and concentrated under reduced pressure to furnish the crude product. This material was purified by column chromatography (4% MeOH/CH₂Cl₂) to give 1-pyrimidin-2-yl-1,2,3,6-tetrahydro-pyridin-4-carboxylic acid methoxy-methyl amide (48 mg, 58%) as white crystals: ¹H NMR (CDCl₃, 400 MHz) δ 8.35 (d, J=6.4 Hz, 2H), 6.53 (t, J=6.4 Hz, 1H), 6.43 (m, 1H), 4.35 (m, 2H), 3.99 (t, J=5.6 Hz, 2H), 3.66 (s, 3H), 3.27 (s, 3H), 2.55 (m, 2H).

E. Biphenyl-4-yl-(1-pyrimidin-2-yl-1,2,3,6-tetrahydro-pyridin-4-yl)-methanone: To a solution of 1-pyrimidin-2-yl-1,2,3,6-tetrahydro-pyridin-4-carboxylic acid methoxy-methyl amide (48 mg, 0.196 mmol) in THF (1 ml), was added 1-biphenyl-4-yl magnesium bromide (0.5 M in THF) at 0° C. The mixture was stirred at this temperature for 1 hour and quenched with addition of water (5 ml) and ethyl acetate (20 ml). The aqueous phase was further extracted with ethyl acetate (2×8 ml). The combined organic layers were washed with brine (5 ml), dried (MgSO₄), filtered, and concentrated under reduced pressure to furnish the crude product. This material was purified by column chromatography (4% MeOH/CH₂Cl₂) to give biphenyl-4-yl-(1-pyrimidin-2-yl-1,2,3,6-tetrahydro-pyridin-4-yl)-methanone (20 mg, 30%) as an off-white solid: ¹H NMR (CDCl₃, 400 MHz) δ 8.38 (d, J=6.4 Hz, 2H), 7.82-7.42 (m, 9H), 6.70 (m, 1H), 6.58 (t, J=6.4 Hz, 1H), 4.51 (m, 2H), 4.13 (t, J=5.6 Hz, 2H), 2.72 (m, 2H); MS calc'd for C₂₂H₂₀N₃O [M+H]⁺: 342; Found: 342.

F. (S/R)-biphenyl-4-yl-(1-pyrimidin-2-yl-1,2,3,6-tetrahydro-pyridin-4-yl)-methanol: To a solution of biphenyl-4-yl-(1-pyrimidin-2-yl-1,2,3,6-tetrahydro-pyridin-4-yl)-methanone in methanol is added an equimolar amount of CeCl₃ heptahydrate and an equimolar amount of sodium borohydride at room temperature. The mixture is stirred for 1 hour and diluted with ethyl acetate. The mixture is washed with water, brine, dried (MgSO₄), filtered, and concentrated under reduced pressure to furnish the crude product. This material is purified by column chromatography to give (S/R)-biphenyl-4-yl-(1-pyrimidin-2-yl-1,2,3,6-tetrahydro-pyridin-4-yl)-methanol.

G. (S)-biphenyl-4-yl-(1-pyrimidin-2-yl-1,2,3,6-tetrahydro-pyridin-4-yl)-methanol: (S/R)-biphenyl-4-yl-(1-pyrimidin-2-yl-1,2,3,6-tetrahydro-pyridin-4-yl)-methanol is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.5. Preparation of (S)-(1-(Pyrimidin-2-yl)piperidin-4-yl)(2′,3,4′-trifluorobiphenyl-4-yl)methanol

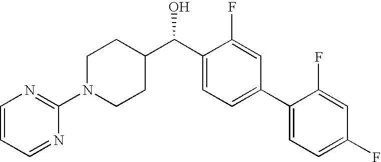

The title compound is isolated by separating the enantiomers of (S/R)-(1-(pyrimidin-2-yl)piperidin-4-yl)(2′,3,4′-trifluorobiphenyl-4-yl)methanol. The racemic mixture was prepared stepwise, as described below.

A. (4-Bromo-2-fluoro-phenyl)-(1-pyrimidin-2-yl-piperidin-4-yl)-methanol: (4-Bromo-2-fluoro-phenyl)-(1-pyrimidin-2-yl-piperidin-4-yl)-methanone was dissolved in 130 ml of EtOH, and then 0.75 ml (23.8 mmol) of hydrazine was added. The mixture was heated to 45° C. with stirring and allowed to proceed to the next day. The reaction mixture was concentrated and diluted with DCM, and then filtered through a thin pad of silica gel. The solvents were evaporated to obtain 2.01 g (90%) of the titled alcohol. LC-MS [M+1] (Column: Shim-Pack VP-ODS 4.6×50 mm)=366.0 (doublet).

B. (S/R)-(1-Pyrimidin-2-yl-piperidin-4-yl)-(3,2′,4′-trifluoro-biphenylyl-4-yl)-methanol: To 250.0 mg (0.685 mmol) of the (4-bromo-2-fluoro-phenyl)-(1-pyrimidin-2-yl-piperidin-4-yl)-methanol dissolved in 12 ml of MeCN was added 129.9 mg (0.822 mmol) of 2,4-difluorophenylboronic acid, 189.0 mg (1.370 mmol) of K₂CO₃, 24 mg (0.034 mmol) of PdCl₂(PPh₃)₂ and 2 ml of water. This mixture was microwaved for 10 min at 140° C. It was diluted with 20 ml of ethyl acetate, washed with water and brine, and then dried over MgSO₄. It was concentrated and purified by preparative HPLC to obtain 204 mg (75%) of (S/R)-(1-(pyrimidin-2-yl)piperidin-4-yl)(2′,3,4′-trifluorobiphenyl-4-yl)methanol. LC-MS [M+1] (Waters ZQ LC/MS, Column: Sunfire C18 5μ 5 cm×4.6 mm ID, Solvent A: acetonitrile; Solvent B: 10 mM ammonium acetate in water)=366.0 (doublet).

C. (S)-(1-Pyrimidin-2-yl-piperidin-4-yl)-(3,2′,4′-trifluoro-biphenyl-4-yl)-methanol: (S/R)-(1-(pyrimidin-2-yl)piperidin-4-yl)(2′,3,4′-trifluorobiphenyl-4-yl)methanol is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.6. Preparation of (S)-(3′-Chloro-3-methylamino-biphenyl-4-yl)-(1-pyrimidin-2-yl-piperidin-4-yl)-methanol

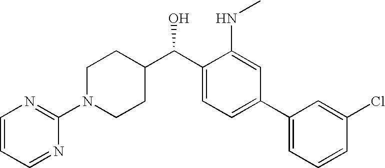

The title compound is isolated by separating the enantiomers of (S/R)-(3′-chloro-3-methylamino-biphenyl-4-yl)-(1-pyrimidin-2-yl-piperidin-4-yl)-methanol. The racemic mixture was prepared stepwise, as described below.

A. (4-Bromo-2-methylamino-phenyl)-(1-pyrimidin-2-yl-piperidin-4-yl)-methanone: To 100 mg (0.275 mmol) of (4-bromo-2-fluoro-phenyl)-(1-pyrimidin-2-yl-piperidin-4-yl)-methanone was added 10.2 mg (0.331 mmol) of H₂NCH₃, 57 mg (0.413 mmol) of K₂CO₃, and 5 ml of DMF. The mixture was heated at 130° C. for 2 hr with stirring. It was then cooled to room temperature, diluted with EtOAc, washed with water and brine and dried over MgSO₄. Solvents were removed and the crude mixture was purified on a preparative TLC plate using 40% EtAc/hex to obtain 90 mg (87%) of the desired product.

B. (S/R)-(3′-chloro-3-methylamino-biphenyl-4-yl)-(1-pyrimidin-2-yl-piperidin-4-yl)-methanol: To a solution of 50 mg (0.123 mmol) of (3′-chloro-3-methylamino-biphenyl-4-yl)-(1-pyrimidin-2-yl-piperidin-4-yl)-methanone in 8 ml of MeOH at 0° C., was added 5.11 mg (0.135 mmol) NaBH₄. The reaction mixture was allowed to stir and warm to room temperature. After 1 hr, LCMS showed that the reaction had gone to completion. It was quenched with water, and the product extracted with EtOAc. This was then subjected to purification by preparative HPLC to obtain the desired product. LC-MS [M+1] (Waters ZQ LC/MS, Column: Sunfire C18 5μ 5 cm×4.6 mm ID, Solvent A: acetonitrile; Solvent B: 10 mM ammonium acetate in water)=409.1 (doublet). HPLC (Discovery Analytical System; Shim-pack VP ODS 4.6×50 mm; Solvent A: Water+0.1% TFA; Solvent B: MeOH+0.1% TFA; start % B=10, final % B=90; wavelength: 220; gradient time: 2 min; flow rate: 3.5 ml/min)=2.17 min.

C. (S)-(3′-chloro-3-methylamino-biphenyl-4-yl)-(1-pyrimidin-2-yl-piperidin-4-yl)-methanol: (S/R)-(3′-chloro-3-methylamino-biphenyl-4-yl)-(1-pyrimidin-2-yl-piperidin-4-yl)-methanol is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.7. Preparation of (S)-(3-Amino-3′-chlorobiphenyl-4-yl)(1-(pyrimidin-2-yl)piperidin-4-yl)methanol

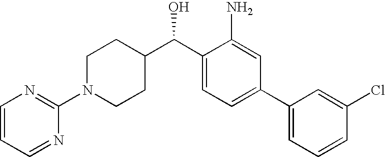

The title compound is isolated by separating the enantiomers of (S/R)-(3-amino-3′-chlorobiphenyl-4-yl)(1-(pyrimidin-2-yl)piperidin-4-yl)methanol. The racemic mixture was prepared stepwise, as described below.

A. [4-Bromo-2-(2,4-dimethoxy-benzylamino)-phenyl]-(1-pyrimidin-2-yl-piperidin-4-yl)-methanone: To 200 mg (0.551 mmol) of (4-bromo-2-fluoro-phenyl)-(1-pyrimidin-2-yl-piperidin-4-yl)-methanone was added 276 mg (1.653 mmol) of 2,4-dimethoxybenzylamine, 304 mg (2.204 mmol) of K₂CO₃ and 15 ml of DMF. This mixture was heated at 130° C. for about 8 hrs. It was cooled to room temperature and diluted with EtOAc, washed with water and brine and dried over MgSO₄. Solvents were removed and the crude mixture was purified by ISCO using 5-40% ethyl acetate/hexanes to obtain 204 mg (67%) of the desired product.

B. (2-Amino-4-bromo-phenyl)-(1-pyrimidin-2-yl-piperidin-4-yl)-methanone: To 204 mg (0.399 mmol) of [4-bromo-2-(2,4-dimethoxy-benzylamino)-phenyl]-(1-pyrimidin-2-yl-piperidin-4-yl)-methanone dissolved in 20 ml of DCM was added 0.92 ml (11.98 mmol, 30.0 equiv) of TFA. The reaction mixture was allowed to stir at room temperature for 20 min. It was concentrated, and residue dissolved 30 ml ethyl acetate. It was washed with NaHCO₃ and brine, dried over MgSO₄ and purified by ISCO, eluting with 1-8% MeOH/DCM to obtain 131 mg (91%).

C. (3-Amino-3′-chloro-biphenyl-4-yl)-(1-pyrimidin-2-yl-piperidin-4-yl)-methanone: To 100 mg (0.278 mmol) of (2-amino-4-bromo-phenyl)-(1-pyrimidin-2-yl-piperidin-4-yl)-methanone dissolved in 4 ml of MeCN was added 52.1 mg (0.33 mmol) of 3-chlorophenylboronic acid, 76.6 mg (0.56 mmol) of K₂CO₃, 9.7 mg (0.014 mmol) of PdCl₂(PPh₃)₂ and 1 ml of water. This mixture was microwaved for 10 min at 140° C. It was diluted with 15 ml of ethyl acetate, washed with water and brine, and then dried over MgSO₄. It was concentrated and purified by preparative HPLC to obtain 94 mg (86%) of the product.

D. (R/S)-(3-Amino-3′-chloro-biphenyl-4-yl)-(1-pyrimidin-2-yl-piperidin-4-yl)-methanone: This compound was obtained using the procedure described in step B of Example 5.6. LC-MS [M+1] (Waters ZQ LC/MS, Column: Sunfire C18 5μ 5 cm×4.6 mm ID, Solvent A: acetonitrile; Solvent B: 10 mM ammonium acetate in water)=395.1 (doublet). HPLC (Discovery Analytical System; Shim-pack VP ODS 4.6×50 mm; Solvent A: Water+0.1% TFA; Solvent B: MeOH+0.1% TFA; start % B=10, final % B=90; wavelength: 220; gradient time: 2 min; flow rate: 3.5 ml/min)=1.94 min.

E. (S)-(3-Amino-3′-chloro-biphenyl-4-yl)-(1-pyrimidin-2-yl-piperidin-4-yl)-methanone: (R/S)-(3-Amino-3′-chloro-biphenyl-4-yl)-(1-pyrimidin-2-yl-piperidin-4-yl)-methanone is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.8. Preparation of (S)—N-(3′-chloro-4-(hydroxy(1-(pyrimidin-2-yl)piperidin-4-yl)methyl)biphenyl-3-yl)acetamide

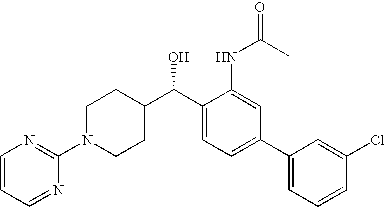

The title compound is isolated by separating the enantiomers of (S/R)—N-(3′-chloro-4-(hydroxy(1-(pyrimidin-2-yl)piperidin-4-yl)methyl)biphenyl-3-yl)acetamide. The racemic mixture was prepared stepwise, as described below.

A. N-[3′-chloro-4-(1-pyrimidin-2-yl-piperidin-4-carbonyl)-biphenyl-3-yl]-acetamide: To 70 mg (0.178 mmol) of (3-amino-3′-chloro-biphenyl-4-yl)-(1-pyrimidin-2-yl-piperidin-4-yl)-methanone dissolved in 15 ml of DCM was added 15.4 mg (0.196 mmol) of the AcCl, and 21.1 mg (0.267 mmol) of pyridine. The reaction mixture was allowed to stir for 2 hr. It was concentrated, and the residue dissolved in 30 ml ethyl acetate, and washed with aq. NaHCO₃. The organic layer was separated, and the aqueous layer extracted twice with 20 ml portions of ethyl acetate. The combined organic layer was washed with brine, and dried over MgSO₄. It was concentrated and the crude mixture was purified by preparative HPLC to obtain 42 mg (54%) of the desired product.

B. (S/R)—N-(3′-chloro-4-(hydroxy(1-(pyrimidin-2-yl)piperidin-4-yl)methyl)biphenyl-3-yl)acetamide: This compound was obtained using the procedure described in step B of Example 5.6. LC-MS [M+1] (Waters ZQ LC/MS, Column: Sunfire C18 5μ 5 cm×4.6 mm ID, Solvent A: acetonitrile; Solvent B: 10 mM ammonium acetate in water)=437.2. HPLC (Discovery Analytical System; Shim-pack VP ODS 4.6×50 mm; Solvent A: Water+0.1% TFA; Solvent B: MeOH+0.1% TFA; start % B=10, final % B=90; wavelength: 220; gradient time: 2 min; flow rate: 3.5 ml/min)=2.11 min.

C. (S)—N-(3′-chloro-4-(hydroxy(1-(pyrimidin-2-yl)piperidin-4-yl)methyl)biphenyl-3-yl)acetamide: (S/R)—N-(3′-chloro-4-(hydroxy(1-(pyrimidin-2-yl)piperidin-4-yl)methyl)biphenyl-3-yl)acetamide is dissolved in a suitable solvent (e.g., 60% ethanol in hexanes). Its enantiomers are separated by normal phase chiral chromatography at ambient temperature using, for example, a ChiralPak AD-H, 20×250 mm column.

### 5.9. Preparation of Additional Compounds

Some racemic compounds, which were prepared by methods analogous to those described above, are listed below in Table 1. The enantiomers of these compounds can be obtained by methods known in the art and described herein.

TABLE 1

| | LCMS | HPLC |
|---|---|---|
| Compound | [M + 1] | [min] |
| (R/S)-N-(3′-Chloro-4-[hydroxyl-(1-pyrimidin-2-yl- | 437.2 | 2.11 |
| piperidin-4-yl)-methyl]-biphenyl-3-yl}-acetamide | | |
| (R/S)-3′-Chloro-4-[hydroxy-(1-pyrimidin-2-yl- | 396.1 | 2.19 |
| piperidin-4-yl)-methyl]-biphenyl-3-ol | | |
| (R/S)-(3′-Chloro-3-methoxy-biphenyl-4-yl)-(1- | 410.1 | 2.33 |
| pyrimidin-2-yl-piperidin-4-yl)-methanol | | |

LC-MS data was obtained under the following conditions: Waters ZQ LC/MS, Column: Sunfire C18 5μ 5 cm×4.6 mm ID, Solvent A: acetonitrile; Solvent B: 10 mM ammonium acetate in water. HPLC data was obtained using the following conditions: Discovery Analytical System; Shim-pack VP ODS 4.6×50 mm; Solvent A: Water+0.1% TFA; Solvent B: MeOH+0.1% TFA; start % B=10, final % B=90; wavelength: 220; gradient time: 2 min; flow rate: 3.5 ml/min.

### 5.10. Human Proline Transporter Assay

The ability of compounds to inhibit the proline transporter was determined as follows. A human SLC6A7 cDNA was cloned into a pcDNA3.1 vector and transfected into COS-1 cells. A cell clone stably expressing proline transporter was selected for the assay.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl, 1.2 mM MgSO₄, 1.2 mM KH₂PO₄, 10 mM HEPES and 5 mM Tris. The cells were then incubated with 50 μl of KRHT buffer containing 45 nM ³H-Proline for 20 minutes at room temperature. Radiolabeled proline uptake was terminated by removing the radiolabeled proline and washing the cells rapidly three times with 100 μl of ice-cold KRHT buffer. Scintillation fluid (50 μl) was added per well, and the amount of tritiated proline present was determined using a Packard TopCount Scintillation counter.

Nonspecific uptake was determined by measuring of ³H-proline uptake in the presence of 2 mM cold proline.

The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The IC₅₀ of a compound was determined using the ten data points, each of which was an average of the four corresponding measurements.

### 5.11. Murine Proline Transporter Assay

Forebrain tissue was dissected from a wild type mouse and homogenized in 7 ml ice-cold homogenization buffer: 0.32 M sucrose, 1 mM NaHCO₃, protease inhibitor cocktail (Roche).

The brain homogenates were centrifuged at 1000×g for 10 min to remove nuclei. Supernatant was collected and re-centrifuged at 20000×g for 20 min to pellet crude synaptosomes. The synaptosomes were resuspended in ice-cold assay buffer: 122 mM NaCl, 3.1 mM KCl, 25 mM HEPES, 0.4 mM KH₂PO₄, 1.2 mM MgSO₄, 1.3 mM CaCl₂, 10 mM dextrose at pH 7.4. Resuspended synaptosomes were centrifuged again at 20000×g for 20 minutes, and pelleted synaptosomes were resuspended in assay buffer. Protein concentration was measured by DC protein assay kit (BioRad).

Proline transport assay was performed in 100 μl reaction mix consisting of 10 μg synaptosomes, 1 μCi/0.24 μM [H3]-proline in assay buffer for a time between 0 to 20 minutes at room temperature. The reaction was terminated by rapid filtration through GF/B filter plate (Millipore) followed by three rapid washes in 200 ul ice-cold assay buffer. Fifty microliters of Microscint-20 was added to each reaction and incubated for 2 hours. The [H3]-proline transport was determined by radioactivity counting.

To determine proline transport inhibition by compounds, compounds were incubated with the reaction mixture at concentrations ranging from 0 to 10 μM (11 points, beginning at 10 um; 3-fold dilutions; 4 replicates averaged to provide one point). The baseline activity, or nonspecific activity, was measured in the presence of 0.3 mM GGFL (Enkephalin, Sigma) in the reaction. The nonspecific activity was also measured in synaptosomes of SLC6A7 knockout mice. The nonspecific activities measured by the two methods were found to be identical.

### 5.12. Human Dopamine Transporter Assay

The ability of compounds to inhibit the dopamine transporter was determined as follows. A human DAT cDNA (NM_{—}001044) was cloned into a pcDNA3.1 vector and transfected into COS-1 cells. The resulting cell lines that stably express the dopamine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 125 mM NaCl, 4.8 mM KCl, 1.3 mM CaCl₂, 1.2 mM MgSO₄ 10 mM D-glucose, 25 mM HEPES, 1 mM sodium ascorbate and 1.2 mM KH₂PO₄. The cells were then incubated with 50 μl of KRHT buffer containing 1 μM ³H-Dopamine for 10 minutes at room temperature. Radiolabeled dopamine uptake was terminated by removing the radiolabeled dopamine and washing the cells rapidly three times with 100 μl of ice-cold KRHT buffer. Scintillation fluid (50 μl) was added per well and the amount of tritiated dopamine present was determined using a Packard TopCount Scintillation counter.

Nonspecific uptake was determined by measuring of ³H-dopamine uptake in the presence of 250 μM benztropine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 5.13. Human Glycine Transporter Assay

The ability of compounds to inhibit the glycine transporter was determined as follows. A human glycine transporter cDNA (NM_{—}006934) was cloned into a pcDNA3.1 vector and transfected into COS-1 cells. The resulting cell lines that stably express the glycine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl₂, 1.2 mM MgSO₄, 1.2 mM KH₂PO₄, 10 mM HEPES and 5 mM Tris. The cells were then incubated with 50 μl of KRHT buffer containing 166 nM ³H-glycine for 10 minutes at room temperature. Radiolabeled glycine uptake was terminated by removing the radiolabeled glycine and washing the cells rapidly three times with 100 μl of ice-cold KRHT buffer. Scintillation fluid (50 μl) was added per well and the amount of tritiated glycine present was determined using a Packard TopCount Scintillation counter.

Nonspecific uptake was determined by measuring ³H-glycine uptake in the presence of 2 mM cold glycine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 5.14. Calculating IC₅₀ Values

The IC₅₀ of a compound with regard to a given target is determined by fitting the relevant data, using the Levenburg Marquardt algorithm, to the equation:

y=A+((B−A)/(1+((C/x)̂D)))

wherein A is the minimum y value; B is the maximum y value; C is the IC₅₀; and D is the slope. The calculation of the IC₅₀ is performed using XLFit4 software (ID Business Solutions Inc., Bridgewater, N.J. 08807) for Microsoft Excel (the above equation is model 205 of that software).

Each of the references (e.g., patents and patent applications) cited herein is incorporated herein in its entirety.

What is claimed is:

1. A stereomerically pure compound of formula I: 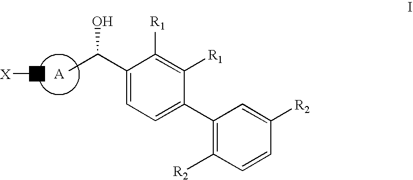
or a pharmaceutically acceptable salt thereof, wherein:
A is an optionally substituted piperidine;
X is optionally substituted pyrimidine;
each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R_{A} is independently hydrogen or optionally substituted alkyl, aryl, arylalkyl, alkylaryl, heterocycle, heterocycle-alkyl, or alkyl-heterocycle; and
each R_{B} is independently hydrogen or optionally substituted alkyl, aryl, arylalkyl, alkylaryl, heterocycle, heterocycle-alkyl, or alkyl-heterocycle.

2. The compound of claim 1, wherein A is unsubstituted.

3. The compound of claim 1, wherein X is of the formula: 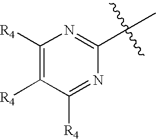
wherein:
each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}.

4. The compound of claim 1, which is of the formula: 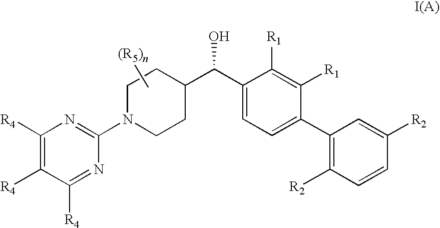
wherein:
each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{A} is independently hydrogen or optionally substituted alkyl, aryl, arylalkyl, alkylaryl, heterocycle, heterocycle-alkyl, or alkyl-heterocycle;
each R_{B} is independently hydrogen or optionally substituted alkyl, aryl, arylalkyl, alkylaryl, heterocycle, heterocycle-alkyl, or alkyl-heterocycle;
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
n is 0-5.

5. The compound of claim 1, which is:
(S)-2-(4-((3′-chlorobiphenyl-4-yl)(hydroxy)methyl)piperidin-1-yl)pyrimidin-5-ol;
(S)-(3′-chlorobiphenyl-4-yl)(1-(pyrimidin-2-yl)piperidin-4-yl)methanol;
(S)-(3-amino-3′-chlorobiphenyl-4-yl)(1-(pyrimidin-2-yl)piperidin-4-yl)methanol;
(S)—N-(3′-chloro-4-(hydroxy(1-(pyrimidin-2-yl)piperidin-4-yl)methyl)biphenyl-3-yl)acetamide;
(S)—N-{3′-chloro-4-[hydroxyl-(1-pyrimidin-2-yl-piperidin-4-yl)-methyl]-biphenyl-3-yl}-acetamide; or
(S)-3′-chloro-4-[hydroxy-(1-pyrimidin-2-yl-piperidin-4-yl)-methyl]-biphenyl-3-ol.

6. A method of improving the cognitive performance of a human patient, which comprises administering to the patient an amount of a compound of claim 1 sufficient to improve the cognitive performance.

7. A method of treating or managing a disease or disorder in a patient, which comprises administering to the patient a therapeutically or prophylactically effective amount of a compound of claim 1, wherein the disease or disorder is age-associated memory impairment, Alzheimer's disease, Attention-Deficit/Hyperactivity Disorder, autism, Down syndrome, Fragile X syndrome, Huntington's disease, Parkinson's disease, or schizophrenia.
